(19) United States
(12) Patent Application Publication
(10) Pub. No.: US 2025/0368549 A1
(43) Pub. Date: Dec. 4, 2025

(54) STERILIZATION PURIFICATION SYSTEM OF CONTAMINATED WATER USING A PLASMA GENERATOR

(71) Applicant: Uniplatek Co., Ltd, Daejeon-si (KR)

(72) Inventor: Suk Hwan KANG, Daejeon-si (KR)

(73) Assignee: Uniplatek Co., Ltd, Daejeon-si (KR)

(21) Appl. No.: 18/679,374

(22) Filed: May 30, 2024

(30) Foreign Application Priority Data
May 30, 2024 (KR) 10-2024-0070441

(51) Int. Cl. C02F 1/30; A61L 2/14; A61L 2/18; A61L 2/24; A61L 2/26; C02F 1/00; C02F 1/46; C02F 1/76; H05H 1/24

(52) U.S. Cl. CPC C02F 1/30; A61L 2/14; A61L 2/18; A61L 2/24; A61L 2/26; C02F 1/001; C02F 1/008; C02F 1/4608; C02F 1/76; H05H 1/2406; A61L 2202/11; A61L 2202/14; C02F 2209/001; C02F 2209/003; C02F 2209/29; C02F 2303/04; H05H 2245/20; H05H 2245/36

(57) ABSTRACT

The present invention relates to a sterilization purification system of contaminated water using a plasma generator, and more specifically, to a sterilization purification system of contaminated water using a plasma generator comprising a contaminated water tank(110) in which contaminated water is stored; a pump(120) that transfers the contaminated water to the plasma generator(150) through the transfer line(140); a filter(130) which is located between the pump(120) and the plasma generator(150) and filters the contaminated water; a plasma generator(150) that generates plasma in low-temperature water to sterilize and purify the contaminated water; a power supply device 160) that supplies power to the plasma generator(150); a purified water tank(180) that stores purified water treated in the plasma generator(150); a first oxidant measuring unit(170) which is located between the plasma generator(150) and the purified water tank(180) and measures the concentration of the oxidant contained in the purified water discharged from the plasma generator (150); a water quality detection sensor that measures the water quality of the contaminated water contained in the contaminated water tank(110) and the water quality of the purified water contained in the purified water tank(180); a display unit that displays the water quality state of the contaminated water and purified water measured in the water quality detection sensor; a control unit that controls the operation of the pump(120), the plasma generator(150), the power supply device(160), and the first oxidant measuring unit(170); and a manager terminal that receives the water quality state of the contaminated water and purified water measured in the water quality detection sensor and controls the operation of the sterilization purification system of contaminated water.

### TECHNICAL FIELD

The present invention relates to a sterilization purification system of contaminated water using a plasma generator, and more specifically, to a sterilization purification system of contaminated water using a plasma generator comprising a contaminated water tank(110) in which contaminated water is stored; a pump(120) that transfers the contaminated water to the plasma generator(150) through the transfer line(140); a filter(130) which is located between the pump(120) and the plasma generator(150) and filters the contaminated water; a plasma generator(150) that generates plasma in low-temperature water to sterilize and purify the contaminated water; a power supply device(160) that supplies power to the plasma generator(150); a purified water tank(180) that stores purified water treated in the plasma generator(150); a first oxidant measuring unit(170) which is located between the plasma generator(150) and the purified water tank(180) and measures the concentration of the oxidant contained in the purified water discharged from the plasma generator(150); a water quality detection sensor that measures the water quality of the contaminated water contained in the contaminated water tank(110) and the water quality of the purified water contained in the purified water tank(180); a display unit that displays the water quality state of the contaminated water and purified water measured in the water quality detection sensor; a control unit that controls the operation of the pump(120), the plasma generator(150), the power supply device(160), and the first oxidant measuring unit(170); and a manager terminal that receives the water quality state of the contaminated water and purified water measured in the water quality detection sensor and controls the operation of the sterilization purification system of contaminated water.

### BACKGROUND ART

In general, the treatment of contaminated water can be performed using expensive filtration systems, ozone(O₃), ultraviolet(UV), electrolysis, etc., or using chemical agents such as chlorine(Cl₂).

In the case of ballast water used in ships, harmful aquatic organisms in seawater may move by the injection and discharge of ballast water. To prevent the problem of foreign marine organisms entering other countries through ship ballast water and destroying the marine ecosystem, ballast water is purified using methods such as electrolysis, ozone treatment, and ultraviolet sterilization. However, these methods require the use of expensive devices and consume a lot of power.

In the case of water used in swimming pools, sterilization is performed with circular filtration and chlorine, and chlorine injection and circular filtration are usually performed 4 to 6 times a day.

The addition of chlorine has an excellent disinfection effect and has the advantage of being able to easily disinfect large amounts of water, but excessive chlorine addition can cause environmental pollution and cause many side effects to the human body.

The efficiency of the ozone treatment method is lower compared with the installation and management costs, and the ultraviolet irradiation method requires frequent replacement of the irradiation lamp, and water polysaccharides may attach to the wall of the lamp's quartz tube, reducing water treatment efficiency.

In addition, water treatment devices and methods using low-temperature plasma have been introduced, but a separate bubble generator must be provided, which makes the device larger and requires a separate installation space.

Therefore, there is a need to develop technology for a sterilization purification system of contaminated water that can treat a large amount of contaminated water in a short time, is easy to install and maintain, requires a small installation space, and can be operated with low energy.

(Patent Document 1) Korean Registration Publication No. 10-1778609

(Patent Document 2) Korean Registration Publication No. 10-1994188

### SUMMARY OF INVENTION

### Technical Problem

The present invention was developed to solve the problems described above, and the purpose of the present invention is to provide a sterilization purification system of contaminated water that can treat a large amount of contaminated water in a short time, is easy to install and maintain, requires a small installation space, and can be operated with low energy.

Another object of the present invention is to provide a sterilization purification system of contaminated water that can continuously sterilize and purify contaminated water by generating plasma in a low-temperature underwater state using a tubular plasma generator(150).

### Solution to Problem

In order to achieve the above object, the present invention provides a sterilization purification system of contaminated water comprising
a contaminated water tank(110) in which contaminated water is stored;
a pump(120) that transfers the contaminated water to the plasma generator(150) through the transfer line(140);
a filter(130) which is located between the pump(120) and the plasma generator(150) and filters the contaminated water;
a plasma generator(150) that generates plasma in low-temperature water to sterilize and purify the contaminated water;
a power supply device(160) that supplies power to the plasma generator(150);
a purified water tank(180) that stores purified water treated in the plasma generator(150);
a first oxidant measuring unit(170) which is located between the plasma generator(150) and the purified water tank(180) and measures the concentration of the oxidant contained in the purified water discharged from the plasma generator(150);
a water quality detection sensor that measures the water quality of the contaminated water contained in the contaminated water tank(110) and the water quality of the purified water contained in the purified water tank(180);
a display unit that displays the water quality state of the contaminated water and purified water measured in the water quality detection sensor;
a control unit that controls the operation of the pump(120), the plasma generator(150), the power supply device(160), and the first oxidant measuring unit(170); and
a manager terminal that receives the water quality state of the contaminated water and purified water measured in the water quality detection sensor and controls the operation of the sterilization purification system of contaminated water.

In one embodiment of the present invention, the plasma generator(150) includes a tubular housing in which contaminated water moves;
a first electrode located inside the housing; and
a second electrode located inside the housing and between the first electrode and the inner wall of the housing.

In one embodiment of the present invention, the first electrode has a spiral structure, a planar structure, a 90° combined structure, or a spiral-90° combined module structure.

In one embodiment of the present invention, the first electrode is characterized in that an insulating layer is coated on the edge surface of the first electrode.

In one embodiment of the present invention, the second electrode has a mesh shape with an empty interior and is positioned adjacent to the inner wall of the housing at a certain distance.

### Advantageous Effects of Invention

The present invention can provide a sterilization purification system of contaminated water that can treat a large amount of contaminated water in a short time, is easy to install and maintain, requires a small installation space, and can be operated with a small amount of supplied energy.

In addition, the present invention can continuously sterilize and purify contaminated water by generating plasma in a low-temperature underwater state using a tubular plasma generator(150).

### BRIEF DESCRIPTION OF DRAWINGS

FIG. 1 shows the spiral-90° combined module structure of the first electrode of the present invention.

FIG. 2 shows the flow of contaminated water through the first electrode of the present invention.

FIG. 3 shows the second electrode of the present invention.

FIG. 4 shows the plasma generator and power supply device of the present invention.

FIG. 5 shows the sterilization purification system of contaminated water of the present invention.

### DESCRIPTION OF EMBODIMENTS

Advantages and features in embodiments of the present invention and methods for achieving them will become clear by referring to the embodiments described in detail below along with the accompanying drawings. However, the present invention is not limited to the embodiments disclosed below and may be implemented in various different forms. The present embodiments are merely provided to ensure that the disclosure of the present invention is complete and to fully inform those skilled in the art of the scope of the invention. The present invention is only defined by the scope of the claims, and the same reference numerals refer to the same elements throughout the specification.

In describing embodiments of the present invention, if a detailed description of a known function or configuration is judged to unnecessarily obscure the gist of the present invention, the detailed description will be omitted. The terms described below are defined in consideration of functions in the embodiments of the present invention, and may vary depending on the intention or custom of the user or operator. Therefore, the definition should be made based on the contents throughout this specification.

Hereinafter, the sterilization purification system of contaminated water according to the present invention will be described with reference to the attached drawings.

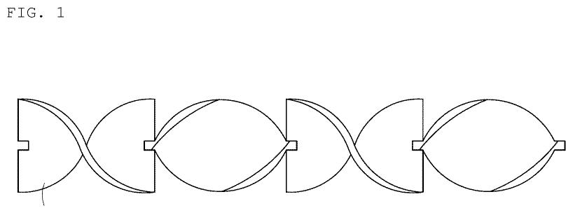

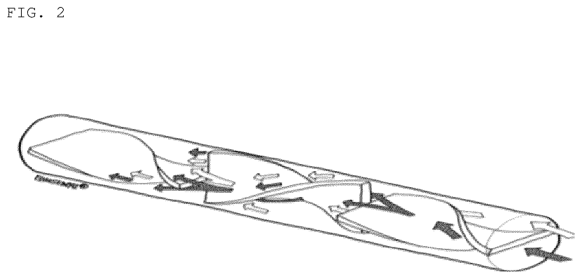

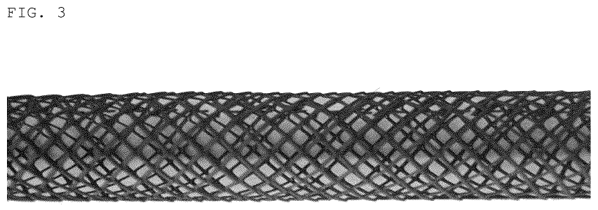

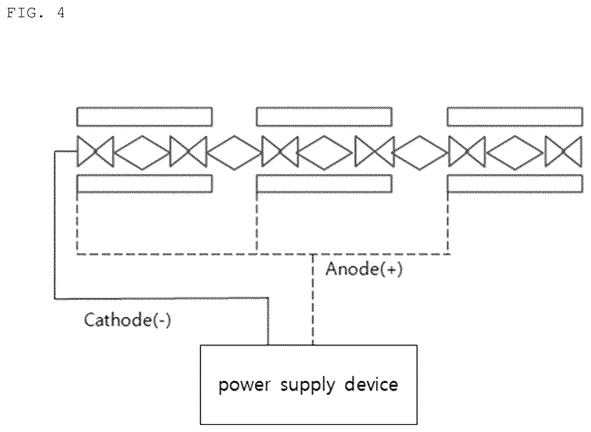

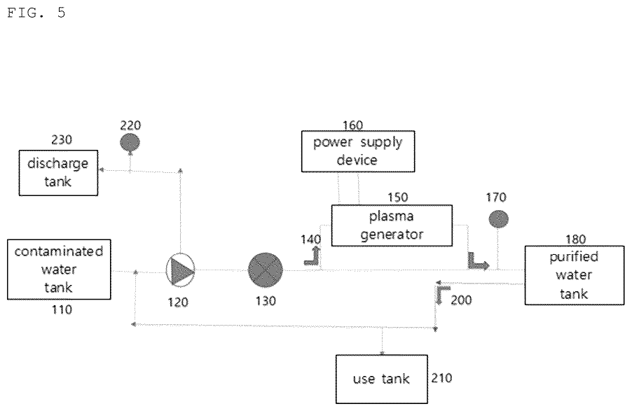

FIG. 1 shows the spiral-90° combined module structure of the first electrode of the present invention, FIG. 2 shows the flow of contaminated water through the first electrode of the present invention, FIG. 3 shows the second electrode of the present invention, FIG. 4 shows the plasma generator and power supply device of the present invention, and FIG. 5 shows the sterilization purification system of contaminated water of the present invention.

Referring to FIGS. 1 to 5, the sterilization purification system of contaminated water of the present invention comprises
a contaminated water tank(110) in which contaminated water is stored;
a pump(120) that transfers the contaminated water to the plasma generator(150) through the transfer line(140);
a filter(130) which is located between the pump(120) and the plasma generator(150) and filters the contaminated water;
a plasma generator(150) that generates plasma in low-temperature water to sterilize and purify the contaminated water;
a power supply device(160) that supplies power to the plasma generator(150);
a purified water tank(180) that stores purified water treated in the plasma generator(150);
a first oxidant measuring unit(170) which is located between the plasma generator(150) and the purified water tank(180) and measures the concentration of the oxidant contained in the purified water discharged from the plasma generator(150);
a water quality detection sensor that measures the water quality of the contaminated water contained in the contaminated water tank(110) and the water quality of the purified water contained in the purified water tank(180);
a display unit that displays the water quality state of the contaminated water and purified water measured in the water quality detection sensor;
a control unit that controls the operation of the pump(120), the plasma generator(150), the power supply device(160), and the first oxidant measuring unit(170); and
a manager terminal that receives the water quality state of the contaminated water and purified water measured in the water quality detection sensor and controls the operation of the sterilization purification system of contaminated water.

The contaminated water tank(110) stores contaminated water, and the contaminated water may include seawater, tap water, swimming pool water, fresh water, cooling tower water, reservoir water, etc.

The pump(120) can transfer the contaminated water to the plasma generator(150) through the transfer line(140), and known pumps can be used without limitation.

The filter(130) is located between the pump(120) and the plasma generator(150) and can filter the contaminated water, and known filters can be used without limitation.

The plasma generator(150) can continuously sterilize and purify the contaminated water by generating plasma in low-temperature underwater state.

The plasma generator(150) includes a tubular housing in which contaminated water moves;
a first electrode located inside the housing; and
a second electrode located inside the housing and between the first electrode and the inner wall of the housing.

The contaminated water continuously moves inside the tubular housing, and the first electrode can form bubbles in the incoming contaminated water and accelerate the distribution of water(FIG. 2).

When power is applied to the plasma generator(150) from the power supply device(160), the applied power is supplied to the first electrode and the second electrode, and a low-temperature plasma is formed in the water due to discharge by the dielectric barrier between the two electrodes. Bacteria, microorganisms, plankton, harmful substances, pollutants, etc. contained in contaminated water can be sterilized and purified by the formed plasma.

As low-temperature plasma is formed, activation electrons are generated. The generated activation electrons can react with chlorine, hydrochloric acid, sodium chloride, etc. contained in contaminated water to generate hypochlorous acid(HOCl), sodium hypochlorite(NaOCl), etc.

The generated hypochlorous acid(HOCl) and sodium hypochlorite(NaOCl) act as oxidizing agents and can sterilize and purify bacteria, microorganisms, plankton, harmful substances, and pollutants contained in contaminated water.

The first electrode may have a shape such as a spiral structure, a planar structure, a 90° combined structure, a spiral-90° combined module structure, and is preferably a spiral structure or a spiral-90° combined module structure. It is more preferable that the spiral-90° combined module structure is arranged repeatedly (FIG. 1).

If the shape of the first electrode is a spiral structure or a spiral-90° combined module structure, bubble formation and water distribution in contaminated water are activated, and the chemical reaction in water can be maximized by increase of the reaction area within the housing of the plasma generator. As bubble formation and water distribution in contaminated water are activated, the generation and movement of activated electrons are accelerated, thereby improving sterilization and purification effects.

The first electrode is preferably made of iron (Fe) and is located inside the tubular housing, preferably at the center of the interior.

In addition, the first electrode is coated with an insulating layer on the edge surface to prevent oxidation of the first electrode, and to prevent the passage of electricity in water between the first electrode and the second electrode to generate plasma.

Additionally, the first electrode may be coated with an insulating layer on a surface adjacent to the second electrode.

The insulating layer is composed of ceramics, inorganic oxides, polymers, etc., and can be coated by methods such as plating and CVD.

In addition, the ratio of the diameter and length of the first electrode may be 1:2, 1:1, 2:1, etc.

The second electrode has a mesh shape with an empty interior, and may be located adjacent to the inner wall of the housing.

The second electrode is preferably made of titanium(Ti), and the first electrode may be disposed within the mesh-shaped second electrode at a certain distance apart.

Additionally, the plasma generator(150) can adjust plasma treatment time, temperature, flow rate, flow volume, etc. depending on the type and water quality state of contaminated water.

The power supply device(160) can supply power to the plasma generator(150), and energy efficiency can be improved by supplying power using an operational amplifier.

The power supply device(160) can improve energy efficiency by supplying a voltage module applied according to the characteristics of contaminated water using an operational amplifier.

Since the current and voltage must be varied depending on the environment or contaminated water, various power supply devices are required. In the past, all of them were deviceized, but the present invention can proceed with system modularization and standardize supply into a standardized module system.

The power supply device(160) can adjust the applied voltage, current, applied frequency, PWM (pulse width modulation), duty ratio, etc. depending on the type of contaminated water, water quality state, etc.

The purified water tank(180) can store purified water treated in the plasma generator(150), and the stored purified water is stabilized for a certain period of time and then transferred to the use tank(210) through the drainage pipe(200) to be used for the purpose.

The first oxidant measuring unit(170) is located between the plasma generator(150) and the purified water tank(180) and can measure the concentration of the oxidant contained in the purified water discharged from the plasma generator(150).

If the concentration of the oxidant measured by the first oxidant measuring unit(170) is less than the set value, the purified water discharged from the plasma generator(150) is not stored in the purified water tank(180), but the purified water can be transferred to the plasma generator(150) again through circulation to perform sterilization and purification treatment.

By repeating this process, if the concentration of the oxidant measured by the first oxidant measuring unit(170) is higher than a set value, the purified water discharged from the plasma generator(150) can be stored in the purified water tank(180).

The water quality detection sensor can measure the water quality state of the contaminated water contained in the contaminated water tank(110) and the water quality state of the purified water contained in the purified water tank(180).

The water quality detection sensor may include a chlorine detection sensor for detecting chlorine, a gas detection sensor for detecting gas, a VOC (volatile organic compound) detection sensor for detecting volatile organic compounds, a fine dust detection sensor for detecting fine dust, etc.

The display unit may display the water quality state of the contaminated water and purified water measured in the water quality detection sensor.

The manager can monitor the water quality state of contaminated water and purified water in real time through the display unit.

The control unit may control the operation of the pump(120), the plasma generator(150), the power supply device(160), the first oxidant measuring unit(170), and the second oxidant measuring unit(220).

The manager terminal receives the water quality state of the contaminated water and purified water measured in the water quality detection sensor through the communication unit, can monitor the water quality state of the contaminated water and purified water in real time, and can control the operation of the sterilization purification system of contaminated water.

The manager can input the operation information of the sterilization purification system of contaminated water through the manager terminal, and the entered operation information is transmitted to the control unit through the communication unit, and the control unit can control operation of the sterilization purification system of contaminated water based on the received operation information.

After the purified water stored in the purified water tank(180) is stabilized for a certain period of time, it is transferred to the use tank(210) through the drainage pipe(200) and used for its intended purpose, or is discharged into the sea, lake, rivers, streams, reservoirs, etc. through the discharge tank(230).

At this time, the concentration of the oxidant contained in the purified water discharged through the discharge tank(230) must be less than a certain value.

The second oxidant measuring unit(220) is located before the discharge tank(230) and can measure the concentration of the oxidant contained in the purified water discharged from the discharge tank(230).

If the concentration of the oxidant measured in the second oxidant measuring unit(220) is higher than the set value, the neutralizing agent is added to the discharge tank(230) so that the concentration of the oxidant contained in the discharged purified water is less than the set value.

In addition, the present invention may further include a matching unit that matches and stores the type and water quality state of contaminated water; plasma treatment conditions (processing time, temperature, flow rate, flow volume, etc.); application conditions of the power supply device (applied voltage, current, applied frequency, PWM (pulse width modulation), duty ratio, etc.); and the water quality state of the purified water.

In addition, the present invention may further include a condition recommendation unit that recommends plasma treatment conditions and application conditions of the power supply device according to the type and water quality state of the contaminated water introduced into the contaminated water tank(110) based on the information of the matching unit.

The present invention can effectively purify the contaminated water by using the plasma treatment conditions and application conditions of the power supply device based on the information of the condition recommendation unit, when the type and water quality state of the contaminated water introduced into the contaminated water tank(110) are identified.

In addition, the present invention can obtain purified water with the desired water quality state by using the plasma treatment conditions and application conditions of the power supply device based on the information of the condition recommendation unit, when the type and water quality state of the contaminated water introduced into the contaminated water tank(110) is identified and the water quality state of the purified water stored in the purified water tank(180) is set.

### EXPLANATION OF SYMBOLS

110: contaminated water tank
120: pump 130: filter
140: transfer line 150: plasma generator
160: power supply device
170: first oxidant measuring unit
180: purified water tank
200: drainage pipe 210: use tank
220: second oxidant measuring unit
230: discharge tank

1. A sterilization purification system of contaminated water comprising
a contaminated water tank(110) in which contaminated water is stored;
a pump(120) that transfers the contaminated water to the plasma generator(150) through the transfer line(140);
a filter(130) which is located between the pump(120) and the plasma generator(150) and filters the contaminated water;
a plasma generator(150) that generates plasma in low-temperature water to sterilize and purify the contaminated water;
a power supply device(160) that supplies power to the plasma generator(150);
a purified water tank(180) that stores purified water treated in the plasma generator(150);
a first oxidant measuring unit(170) which is located between the plasma generator(150) and the purified water tank(180) and measures the concentration of the oxidant contained in the purified water discharged from the plasma generator(150);
a water quality detection sensor that measures the water quality of the contaminated water contained in the contaminated water tank(110) and the water quality of the purified water contained in the purified water tank(180);
a display unit that displays the water quality state of the contaminated water and purified water measured in the water quality detection sensor;
a control unit that controls the operation of the pump(120), the plasma generator(150), the power supply device(160), and the first oxidant measuring unit(170); and
a manager terminal that receives the water quality state of the contaminated water and purified water measured in the water quality detection sensor and controls the operation of the sterilization purification system of contaminated water.

2. The sterilization purification system of contaminated water according to claim 1,
wherein the plasma generator(150) includes a tubular housing in which contaminated water moves;
a first electrode located inside the housing; and
a second electrode located inside the housing and between the first electrode and the inner wall of the housing.

3. The sterilization purification system of contaminated water according to claim 2,
wherein the first electrode has a spiral structure, a planar structure, a 90° combined structure, or a spiral-90° combined module structure.

4. The sterilization purification system of contaminated water according to claim 3,
wherein the first electrode is coated with an insulating layer on a surface adjacent to the second electrode.

5. The sterilization purification system of contaminated water according to claim 4,
wherein the second electrode has a mesh shape with an empty interior and is positioned adjacent to the inner wall of the housing at a certain distance.
